(19) United States
(12) Patent Application Publication
(10) Pub. No.: US 2022/0106358 A1
(43) Pub. Date: Apr. 7, 2022

(54) CONTINUOUS VIRUS RETENTIVE FILTRATION

(71) Applicant: Regeneron Pharmaceuticals, Inc., Tarrytown, NY (US)

(72) Inventors: Ross Browne, Yorktown Heights, NY (US); Erik Schneider, Pittsburgh, PA (US)

(21) Appl. No.: 17/492,490

(22) Filed: Oct. 1, 2021

Related U.S. Application Data
(60) Provisional application No. 63/087,037, filed on Oct. 2, 2020.
(60) Provisional application No. 63/109,942, filed on Nov. 5, 2020.

(51) Int. Cl. C07K 1/34; C07K 16/06; C07K 1/36; C07K 1/20; B01D 15/32; B01D 61/14; B01D 15/36; B01D 15/38

(52) U.S. Cl. CPC C07K 1/34; C07K 16/065; C07K 1/36; C07K 1/20; B01D 15/327; B01D 61/145; B01D 2315/16; B01D 15/3847; B01D 2311/2626; B01D 2311/2623; B01D 2315/10; B01D 2317/04; B01D 15/362

(57) ABSTRACT

The present application provides methods and systems for viral clearance for purifying an antibody from a sample comprising one or more impurities including viral particles. The method is conducted in a system which includes a hydrophobic interaction chromatography (HIC) column and a virus retentive filtration (VRF) system. The HIC column and the VRF system are connected inline in a continuous processing system, and the VRF system comprises at least two filter trains in parallel.

### CROSS-REFERENCE TO RELATED APPLICATIONS

This application claims priority to and the benefit of U.S. Provisional Patent Application No. 63/087,037, filed Oct. 2, 2020 and U.S. Provisional Patent Application No. 63/109,942, filed Nov. 5, 2020 which are each herein incorporated by references.

### FIELD

The present invention generally pertains to methods and systems for purifying an antibody from a sample comprising one or more impurities including viral particles. The method can be conducted in a continuous processing system which includes a hydrophobic interaction chromatography column and a virus retentive filtration system.

### BACKGROUND

Viral clearance is critical to manufacturing biopharmaceutical products, since biological products are accessible to bacteria, fungi and viruses with the risk of transmitting viral diseases. Global health authorities require evaluation of viral clearance for manufacturing biologics or biotechnology products, since viral contamination can be amplified during the growth of mammalian cell cultures. Effective viral clearance studies are an important part of process validation which is critical to ensure drug safety. Viral contamination can also affect raw materials, cell culture processes, bioreactor and downstream purification processes.

Viral validation studies are designed to document selected operating conditions regarding product quality to assure viral safety. The experimental design of viral clearance studies includes critical characterizations of the manufacturing process to identify significant process parameters to improve understanding of processing conditions and justify selection of worst-case conditions.

There are demands for producing biological products in large commercial scales by converting batch mode processing to continuous processing. Viral safety needs to be addressed for the proposed continuous processing to fulfill regulatory requirements. The processes of virus inactivation or removal include pH treatment, heat treatment, solvent/detergent treatment, filtration or chromatography. Filtration steps are considered to be robust viral clearance steps, since the removal mechanism is based on pore sizes of the filters.

It will be appreciated that a need exists for developing methods and systems to effectively incorporate viral clearance and testing for continuous processing of biological products.

### SUMMARY

This disclosure provides methods and systems for incorporating viral clearance and testing during purification of antibodies from a sample comprising one or more impurities including viral particles in a continuous processing system. The proposed continuous processing system includes a hydrophobic interaction chromatography (HIC) column which is connected to a virus retentive filtration (VRF) system.

This disclosure provides a method for purifying an antibody from a sample comprising one or more impurities including viral particles, the method comprising: providing the sample comprising the antibody, and loading the sample to a HIC column which is connected to a VRF system, wherein the HIC column and the VRF system are connected inline in a continuous processing system, and wherein the VRF system comprises at least two filter trains in parallel. In some exemplary embodiments, the method of the present application further comprises a step of single-pass tangential flow filtration and/or pre-filtration. In some exemplary embodiments, a viral reduction capability of the method of the present application is at least 4 LRV (logarithmic reduction value).

In some aspects, each of the at least two filter trains is scheduled at a time point for priming, equilibration, filtering, flushing, integrity testing, sanitization, neutralization or storage. In some aspects, each of the at least two filter trains is switched on or off based on a volumetric throughput or an endpoint of a pressure and is scheduled to operate at a different time interval. In other aspects, each of the at least two filter trains comprises at least one filter, wherein the filter is media filtration, membrane filtration, functional filtration, chromatographic filtration or size-exclusion filtration. In other aspects, the VRF system is operated under constant flow or constant pressure under externally driven feed flow. In yet other aspects, the VRF system is operated under constant flow between about 10 and about 100 liter/m²/hr (LMH). In some aspects, the VRF system is operated under constant flow at about 90 LMH.

This disclosure, at least in part, provides a continuous processing system for purifying an antibody from a sample comprising one or more impurities including viral particles, the continuous processing system comprising: a hydrophobic interaction chromatography (HIC) column, and a virus retentive filtration (VRF) system; wherein the HIC column and the VRF system are connected inline, wherein the sample is loaded to the HIC column, and wherein the VRF system comprises at least two filter trains in parallel. In some aspects, the continuous processing system of the present application further comprises a single-pass tangential flow filtration and/or pre-filtration. In other aspects, a viral reduction capability of the continuous processing system of the present application is at least 4 LRV (logarithmic reduction value).

In some aspects, each of the at least two filter trains is scheduled at a time point for priming, equilibration, filtering, flushing, integrity testing, sanitization, neutralization or storage. In some aspects, each of the at least two filter trains is switched on or off based on a volumetric throughput or an endpoint of a transmembrane pressure and is scheduled to operate at a different time interval. In other aspects, each of the at least two filter trains comprises at least one filter, wherein the filter is media filtration, membrane filtration, functional filtration, chromatographic filtration or size-exclusion filtration. In other aspects, the VRF system is operated under constant flow or constant pressure under externally driven feed flow. In yet other aspects, the VRF system is operated under constant flow between about 10 and about 100 LMH. In some aspects, the VRF system is operated under constant flow at about 90 LMH.

These, and other, aspects of the invention will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 shows a design of a virus retentive filtration (VRF) system, which can be connected to HIC and single-pass tangential flow filtration (SPTFF) according to an exemplary embodiment. The VRF system can be implemented using one or more small-virus filters to reduce the levels of parvovirus and larger viruses from a process stream by size exclusion according to an exemplary embodiment.

FIG. 2 shows a design of a bench-scale continuous VRF system containing multiple filters, which can be connected to continuous HIC and single-pass tangential flow filtration (SPTFF) according to an exemplary embodiment. The continuous VRF system can be implemented using one or more small-virus filters to reduce the levels of parvovirus and larger viruses from a process stream by size exclusion according to an exemplary embodiment.

FIG. 3 shows a monitoring display of a continuous VRF system comprising two or more filter trains in parallel according to an exemplary embodiment. The filter trains of the continuous VRF system can be subjected to different statuses, such as filtering, buffer flushing, or filter primed according to an exemplary embodiment.

FIG. 4 shows a continuous VRF system for viral clearance, comprising two or more filter trains in parallel according to an exemplary embodiment. Each filter train is scheduled for various steps at specific time points under a continuous VRF control logic, such as the step of buffer priming, equilibration, filtering, buffer flushing or integrity testing according to an exemplary embodiment.

FIG. 5 shows the performance of a processing system for purifying a monoclonal antibody using HIC which was connected inline with a VRF system according to an exemplary embodiment. The performance of this processing system was evaluated based on VRF ΔP (psi) as a function of virus filter throughput (L/m²) for three chromatography cycles according to an exemplary embodiment.

FIG. 6 shows the performance of a VRF system under constant pressure runs indicating 56.6% flux decay at 1000 L/m² throughput with greater than 4.6 LRV according to an exemplary embodiment.

FIG. 7 shows the performance of a VRF system under constant flow runs indicating no detectable increase in pressure (less than 1.5 psi) at 1000 L/m² throughput with greater than 5.2 LRV according to an exemplary embodiment.

FIG. 8 shows the performance of a VRF system under constant pressure according to an exemplary embodiment. Terminal flux decay was measured as a function of various operating conditions.

FIG. 9 shows the performance of a VRF system under constant flow and constant pressure according to an exemplary embodiment. The results were analyzed under constant flow and constant pressure in terms of filter permeability as a function of filter throughput.

FIG. 10 shows examples of designing viral clearance studies to characterize design space for continuous VRF systems according to an exemplary embodiment. Viruses were spiked to the continuous process which included HIC, prefilter and Viresolve® Pro according to an exemplary embodiment.

FIG. 11 shows a data management and control strategy of the continuous VRF system according to an exemplary embodiment.

FIG. 12 shows the design of a stability study of minute virus of mice (MVM) at low pH conditions according to an exemplary embodiment.

FIG. 13 shows the design of a stability study of MVM at high pH conditions according to an exemplary embodiment.

FIG. 14 shows the results of the stability studies of MVM at low and high pH conditions with low or high citrate concentration based on MVM LRF (Log 10) as a function of time point (hours) according to an exemplary embodiment.

### DETAILED DESCRIPTION

Since viral contaminations can be amplified during the growth of mammalian cell culture or introduced through contaminated equipment during processing, evaluation of viral clearance for manufacturing biologics or biotechnology products is important to ensure drug safety. Health authorities have provided guidance to manage patient risk for evaluating whether a step clears virus—by knowing how clearance happens, when steps operate independently of each other, whether their capability is additive or not additive, and knowing what affects performance. The evaluation of viral clearance should include demonstrating removal of a specific model virus for retrovirus-like particles which are inherent in the genome of Chinese hamster ovary (CHO) cells (Anderson et al., Endogenous origin of defective retroviruslike particles from a recombinant Chinese hamster ovary cell line, Virology 181(1): 305-311, 1991).

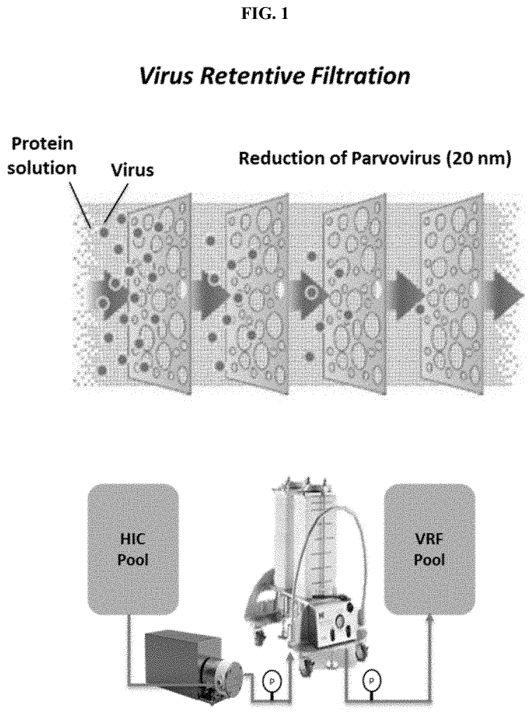

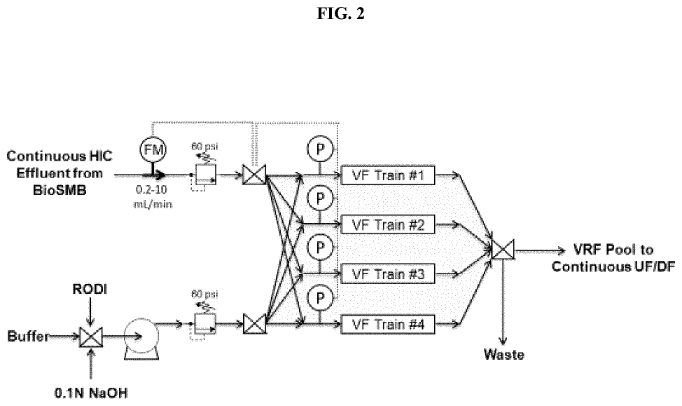

Virus filters are widely used in bioprocessing to reduce the risk of virus contamination in biopharmaceuticals. A non-continuous virus retentive filtration (VRF) system can be connected to a hydrophobic interaction chromatography (HIC) column as shown in FIG. 1. The present application provides a continuous VRF system at bench and manufacturing scale to operate under continuous processing, which can be connected to a HIC column and/or a single-pass tangential flow filtration (SPTFF) system as shown in FIG. 2. The continuous VRF system of the present application can fulfill viral clearance requirements to minimize the likelihood of virus contamination during the manufacture of biopharmaceuticals to satisfy the industrial manufacture and/or regulatory requirements. The continuous VRF system of the present application provides critical quality attributes for viral clearance, such as at least four logarithmic reduction value (LRV). The present application also demonstrates the process of determining critical process parameters and material attributes for the implementation of manufacturing control limits. The continuous VRF system of the present application can be implemented using one or more filters, such as small-virus filters, to reduce the levels of parvovirus and larger viruses from a process stream by size exclusion as shown in FIG. 1. Parvoviruses (parvo meaning small) are a group of very small DNA viruses that are ubiquitous and infect many species of animals. Parvoviruses are non-enveloped, icosahedral particles with diameters of about 18 to 26 nm. The industry expectation of viral clearance when using a small-virus filter step is at least four logs. (John R. Pattison and Gary Patou, Chapter 64 Parvoviruses, Medical Microbiology, 4th edition, Baron S, editor, University of Texas Medical Branch at Galveston, 1996.)

Viral reduction refers to the difference between the total virus amounts in the input sample and output sample after performing a specific process step. The viral reduction capability can be defined as LRV or logarithmic reduction factor (LRF) of a process step. The reduction factor is calculated based on the total virus load before applying the clearance step and the total virus amount after applying the clearance step. Viral validation studies can be conducted to document clearance of known viruses associated with the product and to estimate the effectiveness of the process to clear potential viral contaminants by characterizing the ability of the process to clear non-specific model viruses.

Typical workflow for studying viral clearance of a manufacturing process includes spiking the sample load with virus, running the process on a scale-down experiment to mimic a large-scale step and documenting the ability to clear the spiked virus. Regulatory guidelines recommend using virus validation data to design in-process limits for determining critical process parameters, such as conducting validations at process extremes. Tests can be performed under worst-case conditions to demonstrate the minimum clearance which a process step can provide (1998, Q5A Viral Safety Evaluation of Biotechnology Products Derived from Cell Lines of Human or Animal Origin. T. I. C. f H. o. T. R. f P. f. H. Use). Worst-case conditions can be determined by factors that influence the viral clearance mechanism depending on the process used. The worst-case conditions can be tested to demonstrate the minimal viral reduction of a specific process step (Aranha et al., Viral clearance strategies for biopharmaceutical safety, part II: a multifaceted approach to process validation, BioPharm 14 (5), 43-54, 90, 2001).

Viral validation studies can be designed to document the selected operating conditions regarding product quality and process specificity to assure viral safety. The processes of virus inactivation or removal include pH treatment, heat treatment, filtration or chromatography. Low pH incubation can be used to inactivate enveloped virus, such as by irreversible denaturation of capsid (Brorson et al., Bracketed generic inactivation of rodent retroviruses by low pH treatment for monoclonal antibodies and recombinant proteins, Biotechnol Bioeng 82(3): 321-329, 2003). Filtration is a size-based removal which can be used to remove both enveloped and non-enveloped viruses (Lute et al., Phage passage after extended processing in small-virus-retentive filters, Biotechnol Appl Biochem 47(Pt 3): 141-151, 2007). Chromatography steps can be used to purify biologics products with a potential to provide viral reduction for viral clearance, such as protein A chromatography (Bach et al., Clearance of the rodent retrovirus, XMuLV, by protein A chromatography, Biotechnol Bioeng 112(4): 743750, 2015) or anion exchange chromatography.

There are various challenges to designing a continuous VRF system incorporating viral testing, clearance or inactivation methods for the production of biopharmaceutical products. Constant flow operation must be maintained to conduct continuous VRF, while maintaining constant pressure is the industrial standard. When the continuous VRF system is connected inline with HIC, the operating pressure is limited. For example, when a surge tank, for example, a storage reservoir, is not installed in the inlet, the continuous VRF system will operate at the conditions established by HIC. Various factors can have impacts on the viral clearance including throughput, process fluid flow and handling. The changes in feed material compositions over time also present challenges for conducting continuous VRF. In addition, fluctuations in protein concentrations need to be evaluated during viral clearance studies. (Strauss, et al., Characterizing the Impact of Pressure on Virus Filtration Processes and Establishing Design Spaces to Ensure Effective Parvovirus Removal. Biotechnology Progress, vol. 33, no. 5, 2017, pp. 1294-1302, doi:10.1002/btpr.2506). Some approaches have been adapted to overcome these challenges. For example, continuous processes are performed up to VRF and subsequently batch VRF and batch ultrafiltration/diafiltration (UF/DF) are conducted. Some processes also adapt two virus filters in series to mitigate the risk of integrity test failure. Another option is shifting design space to lower operating pressures or fluxes with longer processing times.

The small pores of the filters for retaining viruses are sensitive to plugging by trace contaminants and frequently require inline adsorptive prefiltration. The issue of clogging and filter overload needs to be overcome to validate continuous viral clearance. Performing integrity testing on every filter used can be a critical strategy to quarantine processed material from adventitious viral contaminants. Integrity testing on the filter after filtering the processed material can detect whether the integrity of the filter has been compromised during the process. Since the continuous culture can be run for longer periods, specific time points should be selected to test for the presence of adventitious agents. There are challenges in quarantining affected material after the observation of failed integrity tests. In some cases, there is no specific flow-decay to indicate the breakthrough of the filter, and validated volumetric throughput becomes the only reliable parameter for determination of when to terminate use of the filter. The consistency of the flow and the fluctuation of the composition of the load material, such as concentration, pH or conductivity, may have impacts on the effectiveness of the continuous VRF system for viral clearance.

Other challenges include process interruptions, such as depressurization, which can occur when the valves are switched between filtration and buffer flush while performing continuous HIC. Studies have been done to investigate the impacts of process interruptions on virus retention of small-virus filters during continuous processing. The studies show that virus retention may be reduced by process interruptions depending on the designs or operating mechanisms of the filters. Some types of filters are more susceptible to process interruptions than others. Process interruptions may cause a reduction of the ability of the filter to retain virus, which may be related to pore-size distribution and the position of the virus within the filter matrix at the time of process interruption. When the convective flow is resumed after the process interruption, the retained virus may have chances to find a path through the filter. (Genest, P., Slocum, A., LaCasse, D., Pizzelli, K., Greenhalgh, P., Mullin, L. Impact of Process Interruption on Virus Retention of Small-Virus Filters. Bioprocess Int. Bioprocess Tech., 11 (10), 34-44).

A scaled-down study can be designed to investigate the viral reduction capability of a continuous VRF system which comprises one or more small-virus filters, process materials and buffers. A scaled-down model should represent the manufacturing process as closely as possible and be operated under representative conditions of the manufacturing scale process. The validated LRV may not be guaranteed to represent the large scale process, if a validation study does not accurately represent the manufacturing process. A parvovirus, such as minute virus of mice (MVM), can be spiked into the process as a model for investigating the scaled-down study of a continuous VRF system. MVM is often the model of choice, since MVM is highly resistant to chemical treatments and has a small particle size, such as 18-24 nm, which is particularly challenging to remove using filtration through size exclusion. (Genest et al.).

Studies have been done to investigate inline spiking methods for virus filter validation using scaled down filters. If pre-filtration removes viruses and interferes with the measurement of virus filter LRV, the standard approach is to batch pre-filter the protein solution, spike with virus, and then virus filter. However, standard viral clearance batch studies may not be representative of actual load conditions. For a number of proteins, batch pre-filtration leads to increased plugging and significantly lower throughputs than inline pre-filtration. One study provides an inline pre-filtration with direct measurement of virus filter removal capabilities, which is tested with three different protein feeds and two different parvovirus filters at two virus injection rates. This inline method can reliably measure LRV at throughputs representative of the manufacturing process. (Lutz, Herbert, et al. “Qualification of a Novel Inline Spiking Method for Virus Filter Validation.” Biotechnology Progress, vol. 27, no. 1, 2010, pp. 121-128., doi:10.1002/btpr.500). Protein concentration gradients should be more accurately modeled by inline virus spiking.

More studies have been done to investigate filter design space for validation of virus filtration in continuous processing applications. One study shows successful viral clearance of bacteriophage PP7 spiked PBS (phosphate-buffered saline) at 2.5 psi on Pegasus™ Prime filter. The study includes eight days of processing with a twenty-four hour process pause at day 7 with loading up to a volumetric throughput of about 7000 L/m². It shows that principles of quality by design (QbD) are important to minimize the risk of a changing design space. It is important to use QbD to characterize design space and confirm viral clearance at all expected operating conditions, for example, flow rate, protein concentration, loading, and process pauses. (McAlister, Morven, Virus Filtration in Continuous Processing: Considerations for Filter Design Space and Validation, BioProcess International Conference and Exhibition. BioProcess International Conference and Exhibition, 27 Sep. 2017, Boston, Mass.). The pharmaceutical QbD is a systematic approach to develop processes that begins with predefined objectives by emphasizing product understanding, process understanding and process control based on sound science and quality risk management. Design space verification is a demonstration that the proposed combination of input process parameters and material attributes are capable of manufacturing quality product at commercial scale.

Various design strategies have been proposed to assure viral safety of conducting continuous processing of biological products including an inline virus spiking system. An automated parallel switching system was provided, including a switch-in and switch-out filtration scheme by switching between old and fresh filters before reaching their validated total volumetric throughputs. A proof-of-concept study shows that this automated parallel flow filtration system was able to clear bacteriophage models at greater than or equal to 4 LRVs with and without protein content using traditional batch viral spiking methodologies. Therefore, as long as there is sufficient and robust scientific evidence to provide validated viral clearance and other safety assurances in novel manufacturing schemes, there is no reason not to implement continuous processing (Johnson, Sarah A., et al., Adapting Viral Safety Assurance Strategies to Continuous Processing of Biological Products, Biotechnology and Bioengineering, vol. 114, no. 6, 2017, pp. 1362-1362).

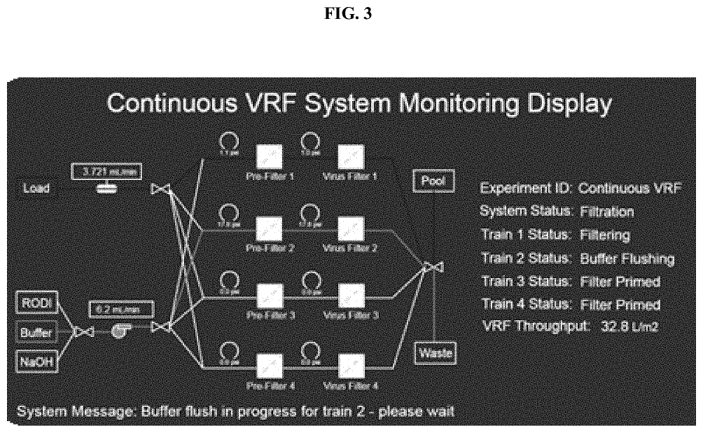

In some exemplary embodiments, the present application provides a continuous VRF system for viral clearance, comprising two or more filter trains in parallel, wherein the continuous VRF system is operated under continuous processing, wherein the filter trains are switched on or off based on a volumetric throughput or an endpoint of a pressure and wherein each filter train comprise one or more filters. In some aspects, the continuous VRF system is connected to HIC, wherein a sample comprising antibodies, viral particles, and one or more impurities can be loaded to the HIC. In one aspect, the continuous VRF system of the present application comprises multiple filter trains in parallel and is operated under externally driven feed flow, such as Cadence® BioSMB System (purchased from Pall Corporation). The continuous VRF system of the present application has the abilities of priming, sanitizing, neutralizing, equilibrating and flushing the filters. For example, as shown in FIG. 3 regarding the monitoring display of a continuous VRF system, the filter train can be subjected to different statuses, such as filtering, buffer flushing, or filter primed. As an example, the system message shows the status of filter trains, such as buffer flush in progress for train 2.

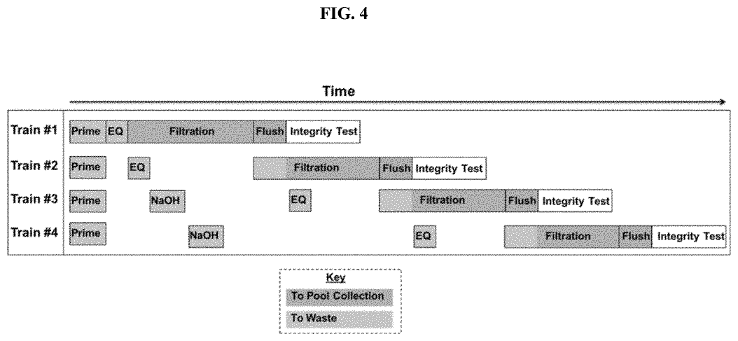

In some exemplary embodiments, the present application provides a continuous VRF system for viral clearance, comprising two or more filter trains in parallel, wherein the continuous VRF system is operated under continuous processing by switching on or off two or more filter trains. Each filter train is scheduled for various steps at specific time points under a continuous VRF control logic, such as the step of buffer priming, equilibration, sanitization, filtering, buffer flushing or integrity testing as shown in FIG. 4. In addition, the continuous VRF system of the present application has a flexible design which enables the implementation of any continuous normal flow filtration step, such as media filtration, membrane filtration or Emphaze™ filtration (Emphaze™ purifier from 3M, Inc). In addition, automated filtration systems can be adapted for other continuous normal flow filtration steps, such as depth filtration, virus filtration of media, or sterile filtration between unit operations.

Media filtration systems operate through physical capture of pollutants and/or adsorption of pollutants through chemical reactions. Membrane filtration systems operate through the use of a permeable thin layer of material (e.g., filter membrane material) which retains impurities and targeted pollutants from liquid flow passed through the permeable layer. The removal mechanism of the membrane filtration is the physical blockage of particles by the filter membrane material. Pore size of the membrane filtration systems refers to the size of the holes or gaps in the filter membrane material. When the pore size of the filter membrane is smaller, smaller particles can be blocked from passing through the filter membrane material. Emphaze™ filtration can be conducted using Emphaze™ purifier which contains synthetic functionalized media, anion exchange media and asymmetric bioburden reduction membrane. Emphaze™ purifier provides flow-through chromatographic separation of contaminants or a combination of chromatographic and size-exclusion mechanisms.

The needs of producing biological product in large commercial scales have led to increasing demand for converting batch mode processing to continuous processing. Viral safety needs to be addressed for the proposed continuous processing system to fulfill regulatory requirements. This disclosure provides methods and systems to satisfy the aforementioned needs by providing methods and systems to effectively incorporate viral clearance and testing to the proposed continuous processing system for manufacturing biological products.

Exemplary embodiments disclosed herein satisfy the aforementioned needs by providing methods and systems for purifying an antibody from a sample comprising one or more impurities including viral particles.

The term “a” should be understood to mean “at least one”; and the terms “about” and “approximately” should be understood to permit standard variation as would be understood by those of ordinary skill in the art; and where ranges are provided, endpoints are included.

As used herein, the terms “include,” “includes,” and “including,” are meant to be non-limiting and are understood to mean “comprise,” “comprises,” and “comprising,” respectively.

In some exemplary embodiments, this disclosure provides a method for purifying an antibody from a sample comprising one or more impurities including viral particles, the method comprising: providing the sample comprising the antibody, and loading the sample to a hydrophobic interaction chromatography (HIC) column which is connected to a virus retentive filtration (VRF) system; wherein the HIC column and the VRF system are connected inline in a continuous processing system, and wherein the VRF system comprises at least two filter trains in parallel. In some aspects, this disclosure provides a continuous processing system for purifying an antibody from a sample comprising one or more impurities including viral particles, the continuous processing system comprising: a hydrophobic interaction chromatography (HIC) column, and a virus retentive filtration (VRF) system; wherein the HIC column and the VRF system are connected inline, wherein the sample is loaded to the HIC column, and wherein the VRF system comprises at least two filter trains in parallel.

As used herein, the term “virus particles” includes infectious agents that replicate inside living cells. The virus particle contains RNA or DNA surrounded by a protein shell called a capsid. The capsid protects the interior core which includes the virus genome and viral proteins. When the virus particle binds to the surface of a specific host cell, the viral DNA or RNA is injected into the host cell for viral replication. Eventually, the viral infection is spread to other host cells. Viral particles may be, for example, parvoviruses, such as minute virus of mice (MVM).

As used herein, the term “antibody” refers to immunoglobulin molecules consisting of four polypeptide chains, two heavy (H) chains and two light (L) chains inter-connected by disulfide bonds. Each heavy chain has a heavy chain variable region (HCVR or VH) and a heavy chain constant region. The heavy chain constant region contains three domains, CH1, CH2 and CH3. Each light chain has a light chain variable region and a light chain constant region. The light chain constant region consists of one domain (CL). The VH and VL regions can be further subdivided into regions of hypervariability, termed complementarity determining regions (CDR), interspersed with regions that are more conserved, termed framework regions (FR). Each VH and VL can be composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” is inclusive of, but not limited to, those that are prepared, expressed, created or isolated by recombinant means, such as antibodies or bispecific antibodies isolated from a host cell transfected to express the antibody. An IgG comprises a subset of antibodies.

As used herein, the term “impurities” or “impurity” can include any undesirable protein or viral particle present in the protein biopharmaceutical product. The impurity can include process and product-related impurities. The impurity can further be of known structure, partially characterized, or unidentified. Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables. Product-related impurities (e.g., precursors, certain degradation products) can be molecular variants arising during manufacture and/or storage that do not have properties comparable to those of the desired product with respect to activity, efficacy, and safety. Such variants may need considerable effort in isolation and characterization in order to identify the type of modification(s). Product-related impurities can include truncated forms, modified forms, and aggregates. Truncated forms are formed by hydrolytic enzymes or chemicals which catalyze the cleavage of peptide or disulfide bonds. Modified forms include, but are not limited to, deamidated, isomerized, mismatched S-S linked, oxidized, or altered conjugated forms (e.g., glycosylation, phosphorylation). Modified forms can also include any post-translational modification form. Aggregates include dimers and higher multiples of the desired product. (Q6B Specifications: Test Procedures and Acceptance Criteria for Biotechnological/Biological Products, ICH August 1999, U.S. Dept. of Health and Humans Services).

### Exemplary Embodiments

Embodiments disclosed herein provide methods and systems for purifying an antibody from a sample comprising one or more impurities including viral particles. The method is conducted in a continuous processing system which includes a HIC column and a VRF system.

In some exemplary embodiments, this disclosure provides a method for purifying an antibody from a sample comprising one or more impurities including viral particles, the method comprising: providing the sample comprising the antibody, and loading the sample to a HIC column which is connected to a VRF system; wherein the HIC column and the VRF system are connected inline in a continuous processing system, and wherein the VRF system comprises at least two filter trains in parallel.

In one aspect, a viral reduction capability of the method of the present application is 0.1-0.9 LRV, 0.9-2.1 LRV, 4.6 LRV, 5.2 LRV, 0.1-4 LRV, 0.1-5 LRV, 0.1-6 LRV, 3-6 LRV, 4-5 LRV or at least 4 LRV.

In one aspect, each of the at least two filter trains comprises at least one filter, wherein the filter is media filtration, membrane filtration, functional filtration, chromatographic filtration or size-exclusion filtration. In one aspect, each of the at least two filter trains is scheduled at a time point for priming, equilibration, filtering, flushing, integrity testing, sanitization, neutralization or storage.

It is understood that the system is not limited to any of the aforesaid filters, antibodies, viral particles, HIC, VRF system, prefiltration or SPTFF. The consecutive labeling of method steps as provided herein with numbers and/or letters is not meant to limit the method or any embodiments thereof to the particular indicated order. Various publications, including patents, patent applications, published patent applications, accession numbers, technical articles and scholarly articles are cited throughout the specification. Each of these cited references is incorporated herein by reference, in its entirety and for all purposes. Unless described otherwise, all technical and scientific terms used herein have the same meaning as commonly understood by one of ordinary skill in the art to which this invention belongs.

This disclosure will be more fully understood by reference to the following Examples, which are provided to describe this disclosure in greater detail. They are intended to illustrate and should not be construed as limiting the scope of this disclosure.

### EXAMPLES

### Materials

1. Filtration: Viresolve® Pro, Viresolve® Pro Shield H and Viresolve® Pro Shield are size based filtration membranes for virus removal, which were purchased from EMD Millipore Corporation. Viresolve® Pro Shield H and Viresolve® Pro Shield are prefilters used inline with the Viresolve® Pro filter. Viresolve® Pro Shield H is based on mixed mode adsorptive chemistry, which is caustic stable and low extractable. Viresolve® Pro Shield is based on cation exchange adsorptive chemistry, which is caustic stable and low extractable.

2. Continuous VRF system: A continuous processing system for purifying a monoclonal antibody was designed and built and connected inline with a chromatography system performing a HIC process. The sample containing antibodies was loaded to the HIC column. The flowthrough of the HIC was loaded to the continuous VRF system. The HIC was conducted using a column with 1 cm internal diameter. The continuous VRF system contained two of Viresolve® Pro Shield H (a filter at 3.1 cm²) and two of Viresolve® Pro (a filter at 3.1 cm²).

### Example 1. Inline Connection of HIC and VRF System

Processing for purifying a monoclonal antibody (e.g., MAB1) was conducted using HIC which was connected inline with a VRF system containing two of Viresolve® Pro Shield H and two of Viresolve® Pro as a proof of concept. The sample containing MAB1 was loaded to the HIC column. The flowthrough of the HIC was loaded to the VRF system. This processing was performed under constant flow based on the flow rate of HIC. The VRF flux was 253 LMH (liter/m²/hr) with 15 psi initial ΔP. Three cycles of HIC were conducted with VRF loading of 1160 L/m². Given a predicted flux decay at constant pressure of about 12%, the corresponding predicted pressure increase at constant flow was about 2 psi.

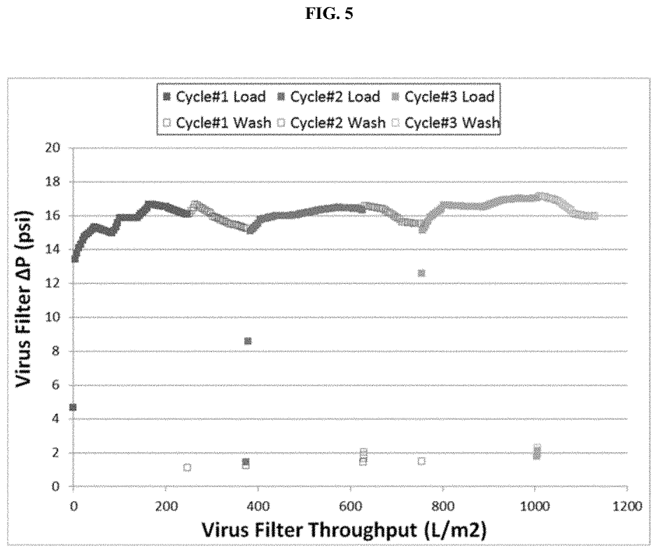

The performance of this processing system was evaluated based on VRF differential pressure as a function of throughput. The differential pressure across the virus filter was plotted as a function of virus filter throughput in L/m² as shown in FIG. 5. The pre-filter inlet pressure during buffer flush was 14.6 psi, the cycle 1 maximum pressure was 16.7 psi, the cycle 2 maximum pressure was 16.6 psi, and the cycle 3 maximum pressure was 17.2 psi. There was no significant increase in filter ΔP observed over the course of constant flow VRF loaded to 1160 L/m², demonstrating the effectiveness of a system with inline connection of HIC and VRF filters. These results were consistent with the performance of previous VRF processes conducted at constant pressure.

### Example 2. Viral Clearance at Constant Flow

Continuous processing for purifying a monoclonal antibody (e.g., MAB2) was conducted with a continuous VRF system. The performance of the VRF system was tested under constant flow and constant pressure modes. The load material included a monoclonal antibody (MAB2) HIC pool that was adjusted to 4.9 pH. Once the load was adjusted to pH 4.9 it was spiked with 0.1% v/v MVM stock. The MVM-spiked material was passed over a 0.1 μm filter to promote a monodispersed virus challenge prior to the virus filter. A low pH was used due to increased HCP clearance by Viresolve® Pro Shield observed during process development. The processing was conducted under constant pressure runs using a compressed nitrogen and pressure can setup, 460 LMH buffer flux at 25 psi, and 1000 L/m² loading. Alternatively, processing was conducted under constant flow runs using AKTA Explorer (a FPLC system purchased from GE Healthcare Life Science, Inc.), 90 LMH at 5 psi initial pressure, and 1000 L/m² loading with post-use buffer flush. The low initial pressure allowed for increased throughput prior to reaching an inlet pressure limit. Low pressure can potentially lead to virus breakthrough due to Brownian motion of virus particles (Strauss Daniel et al.).

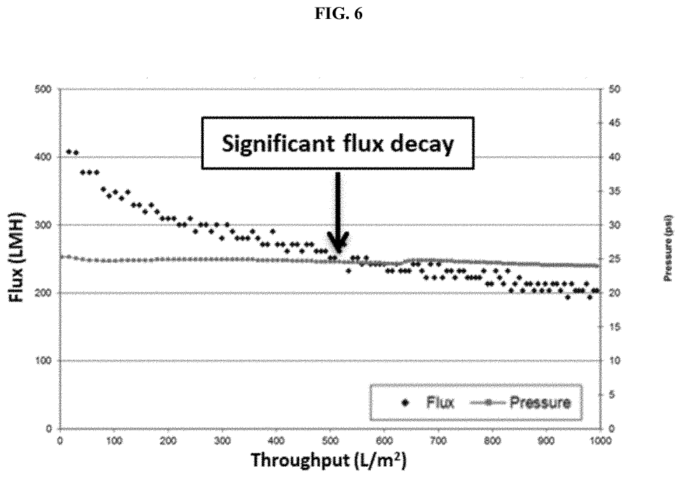

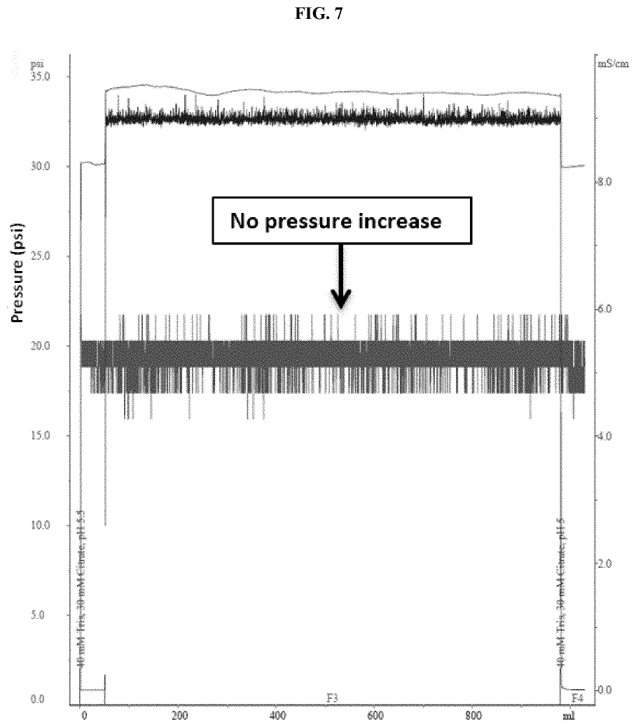

The results under constant pressure runs indicated 56.6% flux decay at 1000 L/m² throughput with greater than 4.6 LRV as shown in FIG. 6. The results under constant flow runs indicated no detectable increase in pressure (less than 1.5 psi) at 1000 L/m² throughput with greater than 5.2 LRV in product pool and buffer flush as shown in FIG. 7. Therefore, 90 LMH flux was sufficient to provide viral clearance. The results also indicated that constant flow runs can result in significantly decreased filter fouling compared to constant pressure runs. The VRF system operated under constant flow mode at low flux of about 90 LMH was able to provide adequate viral safety to enable a fully integrated continuous downstream process.

### Example 3. Comparing Constant Flow and Constant Pressure

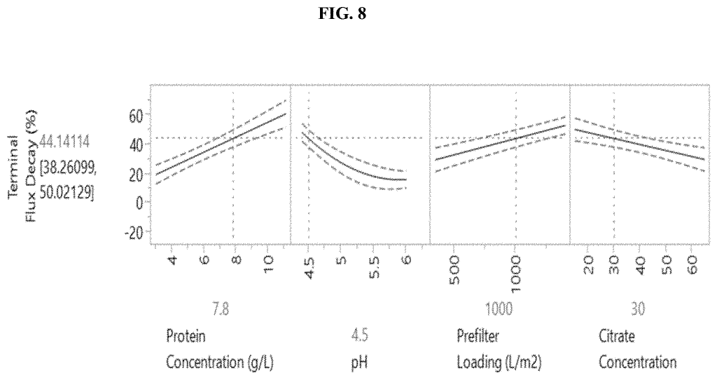

The performance of the continuous VRF system was tested under constant flow and constant pressure modes with conditions that allowed direct comparison. The flow was filtered over Viresolve® Pro and Viresolve® Pro Shield in both constant flow and constant pressure modes on AKTA Explorer (e.g., FPLC) at 270 LMH and 15 psi respectively. A range of conditions were tested to evaluate factors that influenced flux decay in constant pressure mode, as shown in FIG. 8. For the comparison of constant flow and constant pressure modes, the MAB2 HIC pool was adjusted to pH 4.0 to increase fouling, therefore increasing signal. The results were analyzed based on membrane permeability expressed in terms of flux divided by transmembrane pressure (TMP), for example, LMH/psi. The initial permeability was calculated by dividing 270 LMH by 15 psi which was equal to 18 LMH/psi.

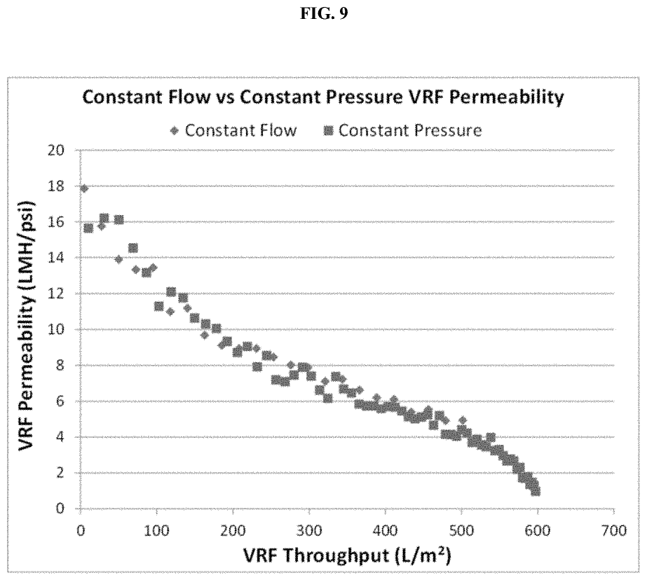

The results under constant pressure runs indicated 92% flux decay at 590 L/m² and 50% permeability decay at 206 L/m² as shown in FIG. 9. The results under constant flow runs indicated 55 psi ΔP at 479 L/m² and 50% permeability decay at 208 L/m². Therefore, the results indicated that constant flow and constant pressure VRF at 270 LMH/15 psi exhibited identical performance in terms of filter permeability as a function of filter throughput. The declines in permeability appeared to be a function of total number of foulant particles removed, which was independent of filtration mode.

### Example 4. Design Features of the Continuous VRF System of the Present Application

A continuous VRF system for viral clearance comprising two or more filter trains in parallel was designed and built as shown in FIG. 3. The continuous VRF system was loaded with a HIC pool sample comprising antibodies, optionally viral particles, and one or more impurities. The continuous VRF system of the present application was run for 3.5 days without interruption in bench scale. The continuous VRF system was operated under continuous processing by switching the filter trains on or off based on a volumetric throughput or an endpoint of a pressure. The continuous VRF system had the abilities of priming, sanitizing, neutralizing, equilibrating and flushing the filters. Each filter train of the continuous VRF system included one or more filters. Each filter train was subjected to different statuses, such as filtering, buffer flushing, or filter primed as shown in FIG. 3. The continuous VRF system had a flexible design to implement various continuous flow filtration steps including media filtration, membrane filtration or Emphaze™ filtration. The continuous VRF system was operated under externally driven feed flow, such as Cadence® BioSMB System. The design of a bench-scale continuous VRF system is shown in FIG. 2.

The testing results showed that choosing pressure versus flow control did not have an impact on the capacity of the continuous VRF system. No virus was detected in constant flow VRF pool operated at about 20% of batch VRF flux. The continuous VRF system connected to an outlet of HIC showed comparable performance to batch VRF.

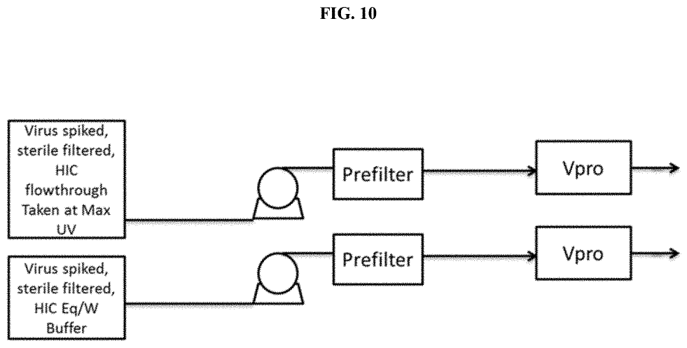

During the operational development of the continuous VRF system and other continuous units, studies were conducted to evaluate the performance of a continuous VRF system at low flow rates including variations of loading molecules, load conditions, protein concentrations, flow rates and process pauses. Prior to transferring first continuous process from development stage to manufacturing, QbD was used to design viral clearance studies to characterize design space for continuous VRF system to minimize the risk of a changing design space. Studies were conducted to investigate filter design space for validation of virus filtration in continuous processing applications. The viral clearance was confirmed at all expected operating conditions. Examples of designing viral clearance studies to characterize design space for continuous VRF system are shown in FIG. 10, with the examples showing alternately taking HIC flowthrough at maximum protein concentration or no protein concentration. Viruses were spiked to the continuous process which included the processing steps of HIC, pre-filter and Viresolve® Pro. Subsequently, the capacity of virus filters for viral clearance were assessed. The filter switching points were programmed into the continuous VRF system based on the minimum throughput achieved in the viral clearance studies.

Each filter train of the continuous VRF system was scheduled for various steps at specific time points under a continuous VRF control logic, such as the step of buffer priming, equilibration, filtering, buffer flushing or integrity testing as shown in FIG. 4. For example, each filter train can be scheduled to operate at a different time interval. The test results indicated that post-use VRF buffer flush enhanced consistency of pool concentration for continuous ultrafiltration/diafiltration (UF-DF). When the virus filters were not in use, they were stored in NaOH as standby filters. The diagram in FIG. 4 is for illustration purposes and the time period for each step is not-to-scale. In particular, the filtration time per filter can be significantly higher compared to traditional batch VRF due to low flux, potentially greater than one day per filter. The process shown in FIG. 4 can be repeated indefinitely, assuming that users intervene in the process to replace spent filters.

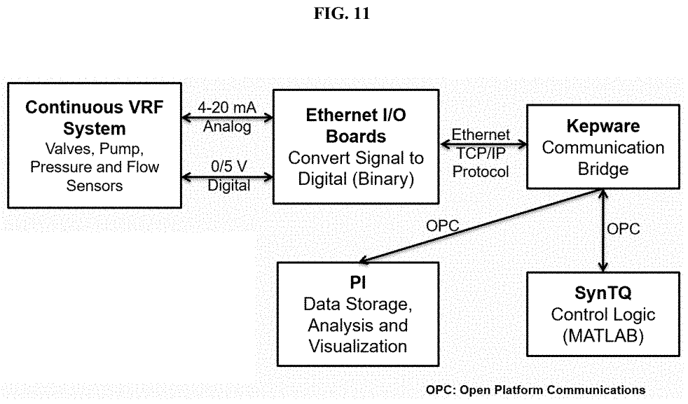

In addition, the design features of the continuous VRF system of the present application included a data management and control strategy as shown in FIG. 11. The continuous VRF system, such as valves, pumps, pressure sensors and flow sensors, communicated directly with Ethernet I/O (input/output) boards. Subsequently Kepware software converted I/O board communication protocol into industry standard OPC (Open Platform Communications). SynTQ has read/write access to Kepware for system control. PI (Process Information) has read-only access to Kepware for data storage, analysis and visualization.

Kepware is a connectivity platform that provides a single source of industrial automation data to applications allowing users to connect, manage, monitor, and control diverse automation devices and software applications through one user interface. OPC is an industrial communication standard that enables the exchange of data between multi-vendor devices and control applications without any proprietary restrictions. PI is a real-time data historian application with a time-series database for recording, analyzing, and monitoring real-time information, such as valves, pumps, flows, pressures or levels. The synTQ PAT Knowledge Management Software Suite can provide universal hardware and software system integration via effective real-time data recording and data management. MATLAB (matrix laboratory) is a high-performance language for technical computing to integrate computation, visualization and programming. Control logic is a key part of a software program that controls the operations of the program.

### Example 5. MVM Stability Studies at Low and High pH Conditions

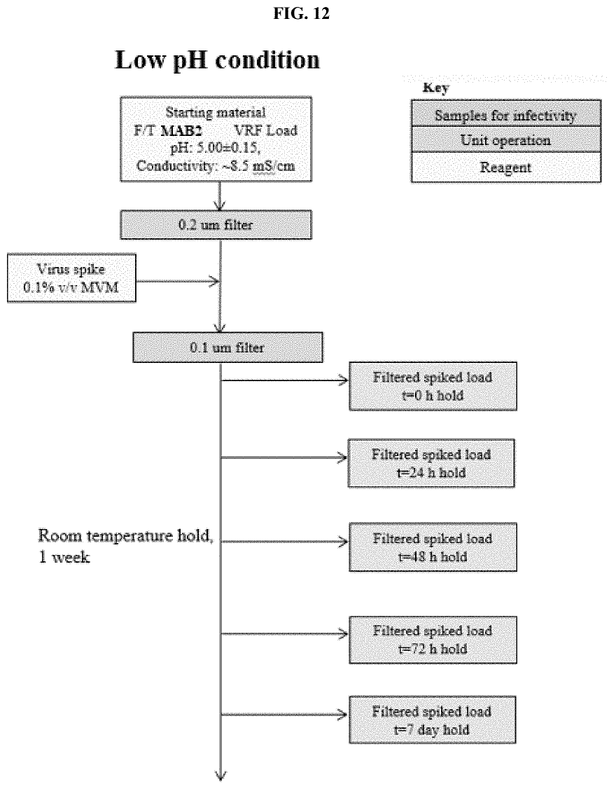

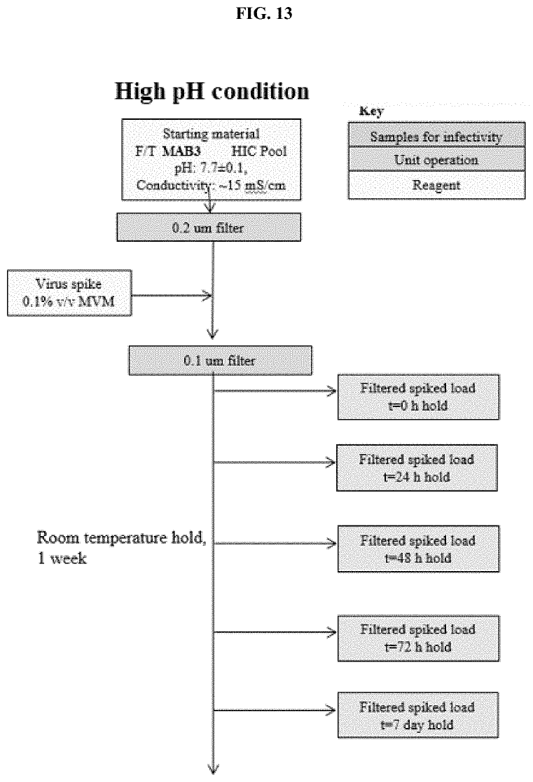

Since virus particles in the VRF load may degrade over time, viral clearance may be an artifact of virus stability without demonstrating actual viral clearance over the filter. The length of time that the spiked virus load is stable will inform how long a single load source can be used for future viral clearance studies. Stability studies of MVM at low and high pH conditions with low or high citrate concentrations were conducted. Examples of the experimental design are shown in FIG. 12 and FIG. 13.

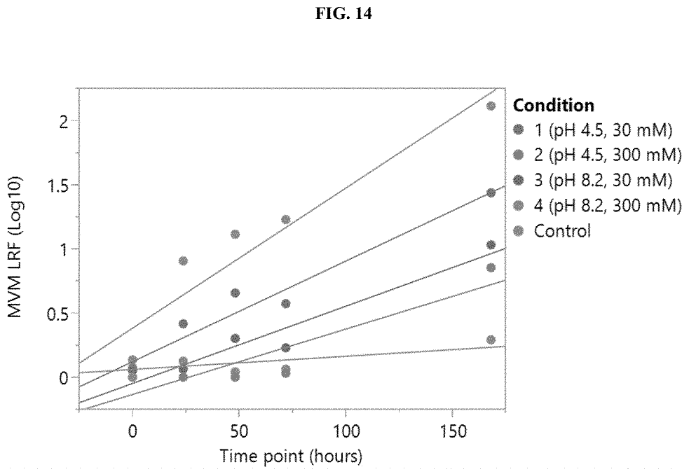

The test results were analyzed based on MVM LRF as a function of time as shown in FIG. 14. Virus stock buffer was used as a control. Virus degradation over time was observed in all evaluated load conditions compared to the control. About 0-0.9 LRF was observed over 24 hours. About 0.9-2.1 LRF was observed over 7 days (168 hours). Based on these test results, load usage may be limited to 24 hours after virus spike to ensure adequate virus load challenge.

What is claimed is:

1. A method for purifying an antibody from a sample comprising one or more impurities including viral particles, the method comprising:
providing the sample including the antibody, and
loading the sample to a hydrophobic interaction chromatography (HIC) column which is coupled to a virus retentive filtration (VRF) system, wherein the HIC column and the VRF system are connected inline in a continuous processing system, and wherein the VRF system comprises at least two filter trains in parallel.

2. The method of claim 1, wherein each of the at least two filter trains is switched on or off based on a volumetric throughput or an endpoint of a pressure.

3. The method of claim 1, wherein the VRF system is operated under constant flow or constant pressure.

4. The method of claim 1, wherein each of the at least two filter trains is scheduled to operate at a different time interval.

5. The method of claim 1, wherein the VRF system is operated under externally driven feed flow.

6. The method of claim 1, wherein each of the at least two filter trains comprises at least one filter, wherein the filter is a media filtration, membrane filtration, functional filtration, chromatographic filtration or size-exclusion filtration.

7. The method of claim 1, wherein each of the at least two filter trains is scheduled at a time point for priming, equilibration, filtration, flushing, integrity testing, sanitization, neutralization or storage.

8. The method of claim 1, wherein the VRF system is operated under constant flow at between about 10 LMH and about 100 LMH.

9. The method of claim 1, wherein the VRF system is operated under constant flow at about 90 LMH.

10. The method of claim 1, wherein the viral reduction capability of the method is at least 4 LRV (logarithmic reduction value).

11. The method of claim 1, further comprising a step of single-pass tangential flow filtration and/or a prefiltration.

12. A continuous processing system for purifying an antibody from a sample comprising one or more impurities including viral particles, the continuous processing system comprising:
a hydrophobic interaction chromatography (HIC) column, and
a virus retentive filtration (VRF) system;
wherein the HIC column and the VRF system are connected inline, wherein the sample is loaded to the HIC column, and wherein the VRF system comprises at least two filter trains in parallel.

13. The continuous processing system of claim 12, wherein each of the at least two filter trains is switched on or off based on a volumetric throughput or an endpoint of a pressure.

14. The continuous processing system of claim 12, wherein the VRF system is operated under constant flow or constant pressure.

15. The continuous processing system of claim 12, wherein each of the at least two filter trains is scheduled to operate at a different time interval.

16. The continuous processing system of claim 12, wherein the VRF system is operated under externally driven feed flow.

17. The continuous processing system of claim 12, wherein each of the at least two filter trains comprises at least one filter, wherein the filter is a media filtration, membrane filtration, functional filtration, chromatographic filtration or size-exclusion filtration.

18. The continuous processing system of claim 12, wherein each of the at least two filter trains is scheduled at a time point for priming, equilibration, filtration, flushing, integrity testing, sanitization, neutralization or storage.

19. The continuous processing system of claim 12, wherein the VRF system is operated under constant flow at between about 10 LMH and 100 LMH.

20. The continuous processing system of claim 12, wherein the VRF system is operated under constant flow at about 90 LMH.

21. The continuous processing system of claim 12, wherein the viral reduction capability of the system is at least 4 LRV (logarithmic reduction value).

22. The continuous processing system of claim 12 further comprising a single-pass tangential flow filtration and/or a prefiltration.
